SEQUENCE LISTING (51 sequences)

SEQ ID NO: 1 - DNA, 27 bp, Homo sapiens
ctgcaatcacgtctgcctgtcacaagt

SEQ ID NO: 2 - DNA, 23 bp, Homo sapiens
ttcagtgagttcatcgtgttcgt

SEQ ID NO: 3 - DNA, 23 bp, Homo sapiens
catcatttgagtcctgccttctc

SEQ ID NO: 4 - DNA, 25 bp, Homo sapiens
ctgctcccctgtttgacgacagtgc

SEQ ID NO: 5 - DNA, 22 bp, Homo sapiens
tttcgacctcgtctttctccat

SEQ ID NO: 6 - DNA, 22 bp, Homo sapiens
tctctccagctcacaggtgaca

SEQ ID NO: 7 - DNA, 26 bp, Homo sapiens
cctttcatcggtgcctggtactctgg

SEQ ID NO: 8 - DNA, 27 bp, Homo sapiens
acattaggaagtggcagttctttactc

SEQ ID NO: 9 - DNA, 20 bp, Homo sapiens
ctcccaccccatcatcattt

SEQ ID NO: 10 - DNA, 22 bp, Homo sapiens
tcggtctgctggtctccgctcc

SEQ ID NO: 11 - DNA, 26 bp, Homo sapiens
ctgattgaagatcattttcaacatca

SEQ ID NO: 12 - DNA, 20 bp, Homo sapiens
gacggccattcagcttatgg

SEQ ID NO: 13 - DNA, 23 bp, Homo sapiens
ctccacccgccagcatccttagc

SEQ ID NO: 14 - DNA, 26 bp, Homo sapiens
tgaattagagcagtctctggtgaaag

SEQ ID NO: 15 - DNA, 23 bp, Homo sapiens
gaatctgacagcgcatcatagaa

SEQ ID NO: 16 - DNA, 21 bp, Homo sapiens
tccccgaacaccaacctgcca

SEQ ID NO: 17 - DNA, 22 bp, Homo sapiens
aaccaccagggaagaaaccatt

SEQ ID NO: 18 - DNA, 21 bp, Homo sapiens
gcataagggctgcagtctgtt

SEQ ID NO: 19 - DNA, 25 bp, Homo sapiens
tgaacacacatgctgccgagctctg

SEQ ID NO: 20 - DNA, 27 bp, Homo sapiens
cttctaggagaacccaacattgatagt

SEQ ID NO: 21 - DNA, 27 bp, Homo sapiens
gtttcttgcaggtacttcttaaaagct

SEQ ID NO: 22 - DNA, 24 bp, Homo sapiens
aaacagccagcctgctacccccag

SEQ ID NO: 23 - DNA, 21 bp, Homo sapiens
ctgccttctctgagcctgaaa

SEQ ID NO: 24 - DNA, 19 bp, Homo sapiens
caaagggcacagagccaaa

SEQ ID NO: 25 - DNA, 24 bp, Homo sapiens
ttgatagaaacgctgtgagctcga

SEQ ID NO: 26 - DNA, 22 bp, Homo sapiens
agctcatcaaggcaattggttt

SEQ ID NO: 27 - DNA, 28 bp, Homo sapiens
acaagatcatgcaagttatcaagaagtt

SEQ ID NO: 28 - DNA, 26 bp, Homo sapiens
agcagcctgcagcctgaagattttgc

SEQ ID NO: 29 - DNA, 24 bp, Homo sapiens
gatctgggacagaattcactctca

SEQ ID NO: 30 - DNA, 19 bp, Homo sapiens
gccgaacgtccaagggtaa

SEQ ID NO: 31 - DNA, 21 bp, Homo sapiens
ccacggccaccgaagaggcac

SEQ ID NO: 32 - DNA, 21 bp, Homo sapiens
cgctcgtgctcatctcctact

SEQ ID NO: 33 - DNA, 24 bp, Homo sapiens
cccagtgcacataaaaggtatgtc

SEQ ID NO: 34 - DNA, 23 bp, Homo sapiens
actgagaatctccacccggcgca

SEQ ID NO: 35 - DNA, 21 bp, Homo sapiens
tcgccaactggataaattgga

SEQ ID NO: 36 - DNA, 22 bp, Homo sapiens
gaatgtgcggaatctgtttgag

SEQ ID NO: 37 - DNA, 28 bp, Homo sapiens
cagatcaggaccaagatggttcccacat

SEQ ID NO: 38 - DNA, 22 bp, Homo sapiens
cattgaagacgcttcgttatgg

SEQ ID NO: 39 - DNA, 27 bp, Homo sapiens
ccagttgtgatggataaaattaatcag

SEQ ID NO: 40 - DNA, 21 bp, Homo sapiens
ccgtcagcctgtgctaggcat

SEQ ID NO: 41 - DNA, 20 bp, Homo sapiens
aatctggaggcaaggttcga

SEQ ID NO: 42 - DNA, 21 bp, Homo sapiens
tctggatttgcctcctgtgaa

SEQ ID NO: 43 - DNA, 24 bp, Homo sapiens
atctccccctacgctctgccagga

SEQ ID NO: 44 - DNA, 23 bp, Homo sapiens
acttgctgtcaattccgagatct

SEQ ID NO: 45 - DNA, 22 bp, Homo sapiens
ggcttgtgctagtgaggtctga

SEQ ID NO: 46 - DNA, 25 bp, Homo sapiens
tggtcagcagcctctctccagtcca

SEQ ID NO: 47 - DNA, 22 bp, Homo sapiens
cgacatctctgcttctcatgct

SEQ ID NO: 48 - DNA, 26 bp, Homo sapiens
agcttgtgtaatagacctccagaaca

SEQ ID NO: 49 - DNA, 23 bp, Homo sapiens
ctctgcgctcctgcatctgcctc

SEQ ID NO: 50 - DNA, 22 bp, Homo sapiens
cgctgtcatcctcattgctact

SEQ ID NO: 51 - DNA, 21 bp, Homo sapiens
tgtggtgtccgaggaatatgg

(19) United States
(12) Patent Application Publication
(10) Pub. No.: US 2016/0348183 A1
(43) Pub. Date: Dec. 1, 2016

(54) METHOD FOR PREDICTING THE RESPONSE AND SURVIVAL FROM CHEMOTHERAPY IN PATIENTS WITH BREAST CANCER

(71) Applicant: MYRIAD GENETICS, INC., Salt Lake City, UT (US)

(72) Inventors: Jan Christoph BRASE, Cologne (DE); Marcus SCHMIDT, Cologne (DE); Ralf KRONENWETT, Cologne (DE); Karin FISCH, Cologne (DE); Karsten WEBER, Cologne (DE)

(73) Assignee: Myriad Genetics, Inc., Salt Lake City, UT (US)

(21) Appl. No.: 15/116,845

(22) PCT Filed: Feb. 11, 2015
(86) PCT No.: PCT/EP2015/052869

§ 371 Date: Aug. 5, 2016

(30) Foreign Application Priority Data
Feb. 12, 2014 (EP) 14154908.9
Mar. 10, 2014 (EP) 14158622.2

(51) Int. Cl. C12Q 1/68

(52) U.S. Cl. CPC C12Q 1/6886; C12Q 2600/118; C12Q 2600/158; C12Q 2600/106; C12Q 2600/112

(57) ABSTRACT

A method predicts the residual risk of recurrence after a taxane-free chemotherapy, and the benefit from inclusion of taxane in a chemotherapy regimen in a patient suffering from or at risk of developing recurrent breast cancer. From determination of expression levels of the genes UBE2C, KIF20A, PTGER3, OSBPL1A, CYP27A1, IGKC, in a tumor sample a prognostic score is determined by mathematically combining the expression level values. The prognostic score is compared to thresholds, classifying the patient in three outcome groups. The expression levels of three genes STC1, PCSK6, S100P in the tumor sample are determined, and the expression level values for STC1, PCSK6 and S100P are mathematically combined to yield a predictive combined score, whereas a high predictive combined score generally indicates an increased likelihood of benefit from inclusion of taxane in a chemotherapy regimen in a patient classified to poor and/or intermediate outcome group.

### TECHNICAL FIELD

The present invention relates to methods, kits and systems for predicting the response and survival from chemotherapy of a breast cancer patient through the analysis of samples from her tumor. More specific, the present invention relates to the prediction of the residual risk of recurrence after standard chemotherapy treatment and the prediction of the response to specific chemotherapeutic agents, in particular to the inclusion of taxane in a chemotherapy regimen based on the measurements of gene expression levels.

### BACKGROUND OF THE INVENTION

Breast cancer is the most common neoplasia in women and remains one of the leading causes of cancer related deaths (Jemal et al., CA Cancer J Clin., 2013). Neoadjuvant or adjuvant chemotherapy is widely used to reduce the risk of recurrence for patients whose clinicopathological risk favors the use of cytotoxic treatment.

Breast cancer is a heterogeneous disease and inherent chemosensitivity differs between molecular breast cancer subtypes. ER− and HER2+ breast tumors are less differentiated, have a high proliferative activity and tend to have a poor prognosis. Therefore, cytotoxic chemotherapy is the standard treatment for both of these subgroups. In contrast to that, clinical management of ER+/HER2− breast cancer patients is challenging, since most of these tumors have a favorable prognosis and only a minority benefits from cytotoxic treatment. Standard clinical parameters (nodal status, tumor size, age, grading) are not appropriate to reliably estimate the likelihood of recurrence and to assist medical-decision making in ER+/HER2− disease. Several prognostic multigene tests have been developed for ER+ breast cancer patients allowing to predicting the risk of recurrence without any chemotherapy treatment and providing a clear answer to the question whether chemotherapy should be used or not (Filipits et al., Clin Can. Res, 2012; Paik et al., NEJM, 2004; Parker et al., JCO, 2009). However, so far chemotherapy regimens not only in ER+/HER2− patients but also in the other subgroups are applied more or less empirically and there is no commercial test currently available that helps to predict response or survival following standard chemotherapy treatment.

Efforts have been taken by Hatzis and colleagues to establish and validate a chemopredictive test for HER2− breast cancer patients using gene expression profiling (Hatzis et al., JAMA, 2011). However, the predictive accuracy of the established signatures has not been validated in an independent cohort as of yet and the huge number of candidate genes causes technical obstacles that could hamper an implementation in clinical routine.

Therefore, there is still a need to establish a chemopredictive test that estimates the risk of recurrence after standard chemotherapy treatment. A large proportion of clinically high risk patients suffer from recurrences despite standard chemotherapy treatment. Patients exhibiting a significant residual risk of recurrence could be encouraged to participate in clinical trials including alternative or extended/intense therapy.

Additionally, little progress has been made in the field of biomarkers that help to select the best chemotherapy regimen for an individual patient. Clinical parameters have little potential to guide the selection of one agent over another.

Anthracyclines and taxanes are among the most effective agents in breast cancer. Several trials demonstrated the effectiveness of these agents when compared to other standard chemotherapy modalities (Martin et al., NEJM, 2005; Gianni et al., JCO, 2009). Taxanes are microtubule stabilizer agents that have been shown to significantly reduce risk of recurrence when compared to standard anthracycline therapy. However, taxanes are generally more toxic and the absolute benefit of taxane-based therapy is moderate and limited to a small percentage of patients.

Since the response to specific chemotherapeutic agents varies considerably among breast cancer patients with cancers exhibiting the same clinical characteristics, identifying patients that are most responsive to taxane-based treatment is still a need to select patients who benefit and to minimize side effects from ineffective therapy in patients without predicted benefit.

Ki-67 has been discussed as a marker to predict taxane efficacy. The PACS01 trial showed that ER+ patients with high Ki-67 expression levels had a particular benefit from taxane-based treatment (Penault-Llorca et al., JCO, 2008). However, the association between Ki67 index and treatment effect has not been validated in an independent breast cancer trial.

Currently, there are no validated predictive markers available to select patients who benefit from taxane-containing therapies. Therefore, there is a great medical need to develop novel biomarkers predicting taxane efficacy and customizing therapy for the individual patient.

### DEFINITIONS

Unless defined otherwise, technical and scientific terms used herein have the same meaning as commonly understood by one of ordinary skill in the art to which this invention belongs.

The term “tumor” as used herein, refers to all neoplastic cell growth and proliferation, whether malignant or benign, and all pre-cancerous and cancerous cells and tissues.

The term “cancer” is not limited to any stage, grade, histomorphological feature, or malignancy of an affected tissue or cell aggregation.

The term “prediction”, as used herein, relates to an individual assessment of the malignancy of a tumor, or to the response to a given therapy, or to the expected survival rate (OAS, overall survival or DFS, disease free survival) of a patient, if the tumor is treated with a given therapy.

A “benefit” from a given therapy is an improvement in health or wellbeing that can be observed in patients under said therapy, but isn't observed in patients not receiving this therapy. Non-limiting examples commonly used in oncology to gauge a benefit from therapy are survival, disease free survival, metastasis free survival, disappearance of metastasis, tumor regression, and tumor remission.

A “risk” is understood to be a probability of a subject or a patient to develop or arrive at a certain disease outcome. The term “risk” in the context of the present invention is not meant to carry any positive or negative connotation with regard to a patient's wellbeing but merely refers to a probability or likelihood of an occurrence or development of a given condition.

A “tumor sample” is a biological sample containing tumor cells, no matter if intact or degraded.

A “gene” is a set of segments of nucleic acid that contains the information necessary to produce a functional RNA product.

An “mRNA” is the transcribed product of a gene or a part of a gene and shall have the ordinary meaning understood by a person skilled in the art.

The term “expression level” refers to a determined level of gene expression. This may be a determined level of gene expression as an absolute value or compared to a reference gene (e.g. a housekeeping gene) or to a computed average expression value (e.g. in DNA chip analysis) or to another informative gene without the use of a reference sample. The expression level of a gene may be measured directly, e.g. by obtaining a signal wherein the signal strength is correlated to the amount of mRNA transcripts of that gene or it may be obtained indirectly at a DNA or protein level, e.g. by immunohistochemistry, CISH, ELISA or RIA methods. The expression level may also be obtained by way of a competitive reaction to a reference sample. An expression value which is determined by measuring some physical parameter in an assay, e.g. fluorescence emission, may be assigned a numerical value which may be used for further processing of information.

As all measurement results also gene expressions values or combined scores, consisting of a mathematical combination of one or more gene expression values, require to be compared to a “reference-value” to get a meaning in a clinical context. As such an expression value or a combined score exceeding such a “reference-value”, by way of example may mean an improved or worsened likelihood of survival for a patient. Such “reference-value” can be a numerical cutoff value, it can be derived from a reference measurement of one or more other genes in the same sample, or one or more other genes and/or the same gene in one other sample or in a plurality of other samples. This is how “reference-value” within the meaning of this invention should be understood.

The term “mathematically combining expression levels”, within the meaning of the invention shall be understood as deriving a numeric value from a determined expression level of at least two genes and combining such determined numerical values by applying an algorithm to obtain a combined numerical value or combined score.

An “algorithm” is a process that performs some sequence of operations to process numerical information.

The term “cytotoxic treatment” or “cytotoxic chemotherapy” refers to various treatment modalities affecting cell proliferation and/or survival. The treatment may include administration of alkylating agents, antimetabolites, anthracyclines, plant alkaloids, topoisomerase inhibitors, and other antitumour agents, including monoclonal antibodies and kinase inhibitors. In particular, the cytotoxic treatment may relate to a treatment comprising microtubule-stabilizing drugs such as taxanes or epothilones. Taxanes are plant alkaloids which block cell division by preventing microtubule function. The prototype taxane is the natural product paclitaxel, originally known as Taxol and first derived from the bark of the Pacific Yew tree. Docetaxel is a semi-synthetic analogue of paclitaxel. Taxanes enhance stability of microtubules, preventing the separation of chromosomes during anaphase. To improve pharmacokinetics and cellular uptake taxanes can be bound to delivery vehicles such as for example albumin (abraxane). Epothilones such as for example Ixabepilone stabilize the microtubules, have the same biological effects and target the same binding site at the microtubule as taxol. However, the chemical structure is different.

The term “neoadjuvant chemotherapy” relates to a preoperative therapy regimen consisting of a panel of hormonal, chemotherapeutic and/or antibody agents, which is aimed to shrink the primary tumor, thereby rendering local therapy (surgery or radiotherapy) less destructive or more effective, enabling breast conserving surgery and evaluation of responsiveness of tumor sensitivity towards specific agents in vivo.

A “taxane” is a drug chemically similar or equivalent to paclitaxel, or docetaxel, or an epothilone, or therapeutically effective derivatives thereof. The principal mechanism of the taxane class of drugs is the disruption of microtubule function. A “taxane-based” treatment or therapy is a treatment, or therapy, or therapy regimen including a taxane. A taxane-free chemotherapy is a chemotherapy not including substances from a class of chemically synthesized or natural compounds called taxanes. Taxanes are substances that cause a disruption of microtubule function.

The term “hybridization-based method”, as used herein, refers to methods imparting a process of combining complementary, single-stranded nucleic acids or nucleotide analogues into a single double stranded molecule. Nucleotides or nucleotide analogues will bind to their complement under normal conditions, so two perfectly complementary strands will bind to each other readily. In bioanalytics, very often labeled, single stranded probes are in order to find complementary target sequences. If such sequences exist in the sample, the probes will hybridize to said sequences which can then be detected due to the label. Other hybridization based methods comprise microarray and/or biochip methods. Therein, probes are immobilized on a solid phase, which is then exposed to a sample. If complementary nucleic acids exist in the sample, these will hybridize to the probes and can thus be detected. These approaches are also known as “array based methods”. Yet another hybridization based method is PCR, which is described above. When it comes to the determination of expression levels, hybridization based methods may for example be used to determine the amount of mRNA for a given gene.

An oligonucleotide capable of specifically binding sequences a gene or fragments thereof relates to an oligonucleotide which specifically hybridizes to a gene or gene product, such as the gene's mRNA or cDNA or to a fragment thereof. To specifically detect the gene or gene product, it is not necessary to detect the entire gene sequence. A fragment of about 20-150 bases will contain enough sequence specific information to allow specific hybridization.

The term “a PCR based method” as used herein refers to methods comprising a polymerase chain reaction (PCR). This is a method of exponentially amplifying nucleic acids, e.g. DNA by enzymatic replication in vitro. As PCR is an in vitro technique, it can be performed without restrictions on the form of DNA, and it can be extensively modified to perform a wide array of genetic manipulations. When it comes to the determination of expression levels, a PCR based method may for example be used to detect the presence of a given mRNA by (1) reverse transcription of the complete mRNA pool (the so called transcriptome) into cDNA with help of a reverse transcriptase enzyme, and (2) detecting the presence of a given cDNA with help of respective primers. This approach is commonly known as reverse transcriptase PCR (rtPCR).Moreover, PCR-based methods comprise e.g. real time PCR, and, particularly suited for the analysis of expression levels, kinetic or quantitative PCR (qPCR).

The terms “Quantitative PCR” (qPCR)” or “kinetic PCR” refers to any type of a PCR method which allows the quantification of the template in a sample. Quantitative real-time PCR comprise different techniques of performance or product detection as for example the TaqMan technique, the LightCycler technique or the usage of a dye directly staining DNA such as SYBR Green. The TaqMan technique, for examples, uses a dual-labelled fluorogenic probe. The TaqMan real-time PCR measures accumulation of a product via the fluorophore during the exponential stages of the PCR, rather than at the end point as in conventional PCR. The exponential increase of the product is used to determine the threshold cycle, CT, i.e. the number of PCR cycles at which a significant exponential increase in fluorescence is detected, and which is directly correlated with the number of copies of DNA template present in the reaction. The set up of the reaction is very similar to a conventional PCR, but is carried out in a real-time thermal cycler that allows measurement of fluorescent molecules in the PCR tubes. Different from regular PCR, in TaqMan real-time PCR a probe is added to the reaction, i.e., a single-stranded oligonucleotide complementary to a segment of 20-60 nucleotides within the DNA template and located between the two primers. A fluorescent reporter or fluorophore (e.g., 6-carboxyfluorescein, acronym: FAM, or tetrachlorofluorescein, acronym: TET) and quencher (e.g., tetramethylrhodamine, acronym: TAMRA, of dihydrocyclopyrroloindole tripeptide “minor groove binder”, acronym: MGB) are covalently attached to the 5′ and 3′ ends of the probe, respectively[2]. The close proximity between fluorophore and quencher attached to the probe inhibits fluorescence from the fluorophore. During PCR, as DNA synthesis commences, the 5′ to 3′ exonuclease activity of the Taq polymerase degrades that proportion of the probe that has annealed to the template (Hence its name: Taq polymerase+TacMan). Degradation of the probe releases the fluorophore from it and breaks the close proximity to the quencher, thus relieving the quenching effect and allowing fluorescence of the fluorophore. Hence, fluorescence detected in the real-time PCR thermal cycler is directly proportional to the fluorophore released and the amount of DNA template present in the PCR.

By “array” or “matrix” an arrangement of addressable locations or “addresses” on a device is meant. The locations can be arranged in two dimensional arrays, three dimensional arrays, or other matrix formats. The number of locations can range from several to at millions. Most importantly, each location represents a totally independent reaction site. Arrays include but are not limited to nucleic acid arrays, protein arrays and antibody arrays. A “nucleic acid array” refers to an array containing nucleic acid probes, such as oligonucleotides, nucleotide analogues, polynucleotides, polymers of nucleotide analogues, morpholinos or larger portions of genes. The nucleic acid and/or analogue on the array is preferably single stranded. Arrays wherein the probes are oligonucleotides are referred to as “oligo-nucleotide arrays” or “oligonucleotide chips.” A “microarray,” herein also refers to a “biochip” or “biological chip”, an array of regions having a density of discrete regions of at least about 100/cm2, and preferably at least about 1000/cm2.

The term “regimen” refers to a timely sequential or simultaneous administration of anti-tumor, and/or anti vascular, and/or immune stimulating, and/or blood cell proliferative agents, and/or radiation therapy, and/or hyperthermia, and/or hypothermia for cancer therapy. The administration of these can be performed in an adjuvant and/or neoadjuvant mode as well in a metastatic setting. The composition of such “protocol” may vary in the dose of the single agent, timeframe of application and frequency of administration within a defined therapy window. Currently various combinations of various drugs and/or physical methods, and various schedules are under investigation.

The term “measurement at a protein level”, as used herein, refers to methods which allow for the quantitative and/or qualitative determination of one or more proteins in a sample. These methods include, among others, protein purification, including ultracentrifugation, precipitation and chromatography, as well as protein analysis and determination, including immunohistochemistry, immunofluorescence, ELISA (enzyme linked immunoassay), RIA (radioimmunoassay) or the use of protein microarrays, two-hybrid screening, blotting methods including western blot, one- and two dimensional gelelectrophoresis, isoelectric focusing as well as methods being based on mass spectrometry like MALDI-TOF and the like.

The term “marker gene” as used herein, refers to a differentially expressed gene whose expression pattern may be utilized as part of a predictive, prognostic or diagnostic process in malignant neoplasia or cancer evaluation, or which, alternatively, may be used in methods for identifying compounds useful for the treatment or prevention of malignant neoplasia and head and neck, colon or breast cancer in particular. A marker gene may also have the characteristics of a target gene.

The term “immunohistochemistry” or IHC refers to the process of localizing proteins in cells of a tissue section exploiting the principle of antibodies binding specifically to antigens in biological tissues. Immunohistochemical staining is widely used in the diagnosis and treatment of cancer. Specific molecular markers are characteristic of particular cancer types. IHC is also widely used in basic research to understand the distribution and localization of biomarkers in different parts of a tissue.

A “score” within the meaning of the invention shall be understood as a numeric value, which is related to the outcome of a patient's disease and/or the response of a tumor to a specific chemotherapy treatment. The numeric value is derived from combining the expression levels of marker genes using pre-specified coefficients in a mathematic algorithm. The expression levels can be employed as CT or delta-CT values obtained by kinetic RT-PCR, as absolute or relative fluorescence intensity values obtained through microarrays or by any other method useful to quantify absolute or relative RNA levels. Combining these expression levels can be accomplished for example by multiplying each expression level with a defined and specified coefficient and summing up such products to yield a score. The score may be also derived from expression levels together with other information, e. g. clinical data like lymph node status or tumor grading as such variables can also be coded as numbers in an equation. The score may be used on a continuous scale to predict the response of a tumor to a specific chemotherapy and/or the outcome of a patient's disease. Cut-off values may be applied to distinguish clinical relevant subgroups. Cut-off values for such scores can be determined in the same way as cut-off values for conventional diagnostic markers and are well known to those skilled in the art.

The term “therapy” refers to a timely sequential or simultaneous administration of anti-tumor, and/or anti vascular, and/or anti stroma, and/or immune stimulating or suppressive, and/or blood cell proliferative agents, and/or radiation therapy, and/or hyperthermia, and/or hypothermia for cancer therapy. The administration of these can be performed in an adjuvant and/or neoadjuvant mode. The composition of such “protocol” may vary in the dose of each of the single agents, timeframe of application and frequency of administration within a defined therapy window. Currently various combinations of various drugs and/or physical methods, and various schedules are under investigation. A “taxane/anthracycline-containing chemotherapy” is a therapy modality comprising the administration of taxane and/or anthracycline and therapeutically effective derivates thereof.

The “response of a tumor to chemotherapy”, within the meaning of the invention, relates to any response of the tumor to cytotoxic chemotherapy, preferably to a change in tumor mass and/or volume after initiation of neoadjuvant chemotherapy and/or prolongation of time to distant metastasis or time to death following neoadjuvant or adjuvant chemotherapy. Tumor response may be assessed in a neoadjuvant situation where the size of a tumor after systemic intervention can be compared to the initial size and dimensions as measured by CT, PET, mammogram, ultrasound or palpation, usually recorded as “clinical response” of a patient. Response may also be assessed by caliper measurement or pathological examination of the tumor after biopsy or surgical resection. Response may be recorded in a quantitative fashion like percentage change in tumor volume or in a qualitative fashion like “no change” (NC), “partial remission” (PR), “complete remission” (CR) or other qualitative criteria. Assessment of tumor response may be done early after the onset of neoadjuvant therapy e.g. after a few hours, days, weeks or preferably after a few months. A typical endpoint for response assessment is upon termination of neoadjuvant chemotherapy or upon surgical removal of residual tumor cells and/or the tumor bed. This is typically three month after initiation of neoadjuvant therapy. Response may also be assessed by comparing time to distant metastasis or death of a patient following neoadjuvant or adjuvant chemotherapy with time to distant metastasis or death of a patient not treated with chemotherapy.

### OBJECT OF THE INVENTION

It is one object of the present invention to provide an improved method for the prediction of residual risk of recurrence after standard chemotherapy treatment in a patient suffering from or at risk of developing a neoplastic disease—in particular breast cancer.

It is another object of the present invention to provide a method for identification of patients, particularly breast cancer patients, who have a benefit from receiving taxanes as a part of their chemotherapy.

It is another object of the present invention to avoid unnecessary side-effects of adjuvant and/or neoadjuvant taxane-based chemotherapy.

It is another object of the present invention to offer a more robust and specific diagnostic assay system than conventional immunohistochemistry for clinical routine fixed tissue samples that better helps the physician to select individualized treatment modalities.

### SUMMARY OF THE INVENTION

This disclosure focuses on a test that predicts the risk of recurrence after a standard chemotherapy treatment and thus will help physicians to decide on regimens and intensity of cytotoxic treatment. Additionally, the test will help to identify patients who will have a benefit from inclusion of taxane in a chemotherapy regimen.

The present invention relates to a method for predicting the residual risk of recurrence after standard chemotherapy treatment, in particular a taxane-free-chemotherapy, and the benefit from inclusion of taxane in a chemotherapy regimen in a patient suffering from or at risk of developing recurrent neoplastic disease, in particular breast cancer. Said method comprises the steps of:

(a) determining in a tumor sample from said patient the expression levels of the following 6 genes: UBE2C, KIF20A, PTGER3, OSBPL1A, CYP27A1, IGKC, and

(b) mathematically combining said expression level values for the genes of the said set which values were determined in the tumor sample to yield a prognostic combined score

and

(c) comparing said prognostic combined score to one or more thresholds and classifying said patient in a good, intermediate or poor outcome group

and

(d) determining in said tumor sample from said patient the expression levels of three genes: STC1, PCSK6, S100P

and

(e) mathematically combining said expression level values for STC1, PCSK6 and S100P to yield a predictive combined score, whereas a high predictive combined score generally indicates an increased likelihood of benefit from inclusion of taxane in a chemotherapy regimen in a patient classified to said poor and/or intermediate outcome group.

According to an aspect of the invention the expression levels of the six genes: KIF20A, UBE2C, PTGER3, OSBPL1A, IGKC and CYP27A1 can be used to calculate a predictive score, whereas a high combined score generally indicates an increased residual risk of recurrence after standard chemotherapy treatment and a low combined score a decreased risk of recurrence after standard chemotherapy treatment.

According to another aspect of the invention the expression levels of three genes: S100P, PCSK6 and STC1 can be used to calculate a predictive score, whereas a high combined score generally indicates an increased likelihood of benefit from inclusion of taxane in a chemotherapy regimen and a low combined score a decreased likelihood of benefit from inclusion taxane in a chemotherapy regimen.

The methods of the invention are particularly suited for predicting residual risk of recurrence after standard chemotherapy treatment and the benefit from including a taxane to cytotoxic chemotherapy, preferably in estrogen receptor positive (ER+), Her2-negative (HER2−) tumors.

According to an aspect of the invention there is provided a method as described above, wherein said expression level is determined as an mRNA level.

According to an aspect of the invention there is provided a method as described above, wherein said expression level is determined by at least one of
a PCR based method,
a microarray based method,
a hybridization based method,
a sequencing and/or next generation sequencing approach

A preferred form is kinetic or quantitative RT-PCR using e.g. commercially available systems such as Taqman, Lightcycler or others.

According to an aspect of the invention there is provided a method as described above, wherein said determination of expression levels is in a formalin-fixed paraffin-embedded tumor sample or in a fresh-frozen tumor sample.

According to an aspect of the invention there is provided a method as described above, wherein the expression level of said marker genes are determined as a pattern of expression relative to at least one reference gene or to a computed average expression value.

According to an aspect of the invention there is provided a method as described above, wherein said step of mathematically combining comprises a step of applying an algorithm to values representative of an expression level of a given gene.

According to an aspect of the invention there is provided a method as described above, wherein said algorithm is a linear combination of said values representative of an expression level of a given gene.

According to an aspect of the invention there is provided a method as described above, wherein a value for a representative of an expression level of a given gene is multiplied with a coefficient.

According to an aspect of the invention there is provided a method as described above, wherein one, two or more thresholds are determined for said combined scores and discriminated into response groups by applying the threshold on the combined score.

According to an aspect of the invention one, two or more thresholds are determined for said gene expression level or combined scores and discriminated into (1) “predicted benefit” and “predicted non-benefit”, (2) “predicted benefit” and “predicted adverse effect”, (3) “predicted benefit”, “predicted indifferent effect” and “predicted adverse effect”, or more response groups with different probabilities of benefit by applying the threshold on the gene expression levels or the combined score.

According to an aspect of the invention there is provided a method as described above, wherein a high combined score is indicative of benefit from a taxane based treatment. The skilled person understands that a “high score” in this regard relates to a reference value or cutoff value. The skilled person further understands that depending on the particular algorithm used to obtain the combined score, also a “low” score below a cut off or reference value can be indicative of benefit from a taxane based therapy.

According to an aspect of the invention there is provided a method as described above, wherein information regarding nodal status of the patient is processed in the step of mathematically combining expression level values for the genes to yield a combined score that predicts residual risk of recurrence after chemotherapy treatment.

The invention further relates to a kit for performing a method as described above, said kit comprising a set of nine oligonucleotides of at least Seq ID Nos: 19, 20 or 21; Seq ID Nos: 16, 17, or 18; Seq ID Nos: 10, 11, or 12; Seq ID Nos: 13, 14, or 15; Seq ID Nos: 25, 26, or 27; Seq ID Nos: 22, 23, or 24; Seq ID Nos: 7, 8, or 9; Seq ID Nos: 4, 5, or 6; and Seq ID Nos: 1, 2, or 3; which oligonucleotides are capable of specifically binding sequences or to sequences of fragments of the genes in a combination of genes, wherein said combination comprises at least the 9 genes UBE2C, KIF20A, PTGER3, OSBPL1A, CYP27A1, IGKC, STC1, PCSK6 and S100P.

Another subject of the present invention is the use of the kit for performing the method of the invention.

The invention further relates to a computer program product capable of processing values representative of an expression level of a combination of genes mathematically combining said values to yield combined scores, wherein said combined scores are predicting said residual risk of recurrence after standard chemotherapy treatment and the benefit from inclusion of taxane in a chemotherapy regimen. The combined scores can be transformed to a given scale in an additional step. Said transformation may be linear or non-linear, continuous or discontinuous, bounded or unbounded, monotonic or non-monotonic.

Said computer program product may be stored on a data carrier or implemented on a diagnostic system capable of outputting values representative of an expression level of a given gene, such as a real time PCR system.

If the computer program product is stored on a data carrier or running on a computer, operating personal can input the expression values obtained for the expression level of the respective genes. The computer program product can then apply an algorithm to produce a combined score predicting said residual risk of recurrence after standard chemotherapy treatment and the benefit from inclusion of taxane in a chemotherapy regimen.

The methods of the present invention have the advantage of providing a reliable prediction of residual risk of recurrence after standard chemotherapy treatment and the benefit from inclusion of taxane in a chemotherapy regimen based on the use of only a small number of genes.

According to an aspect of the invention said cancer is breast cancer. The marker genes described in this invention are not breast cancer specific genes, but generally cancer-relevant genes or genes relevant to the therapeutic mechanism of microtubule stabilizing drugs. It can therefore be expected that the methods of the invention are also predictive in other cancers, in which taxane-based therapy is commonly administered, such as lung cancer, head-and-neck cancer, ovarian cancer and prostate cancer.

### DESCRIPTION OF THE FIGURES

FIG. 1:
a) Receiver operating characteristics (ROC) curve of the exemplary algorithm CP1 in 185 ER+/HER2− breast cancer patients treated with anthracycline-containing chemotherapy. The algorithm combines the gene expression levels of the genes UBE2C, KIF20A, PTGER3, OSBPL1A, CYP27A1 and IGKC. Area under the curve (AUC) is indicated.
b) Kaplan-Meier plot of distant recurrence according to the exemplary algorithm CP1 in 185 ER+/HER2− breast cancer patients treated with anthracycline-containing chemotherapy. The algorithm combines the gene expression levels of the genes UBE2C, KIF20A, PTGER3, OSBPL1A, CYP27A1 and IGKC.

FIG. 2:
a. Receiver operating characteristics (ROC) curve for the of the exemplary algorithm CP1 in 295 ER+/HER2− breast cancer patients treated with taxane/anthracycline-containing chemotherapy. The algorithm combines the gene expression levels of the genes UBE2C, KIF20A, PTGER3, OSBPL1A, CYP27A1 and IGKC. Area under the curves (AUC) is indicated.
b. Kaplan-Meier plot of distant recurrence according to the of the exemplary algorithm CP1 in 295 ER+/ER2− breast cancer patients treated with taxane/anthracycline-containing chemotherapy. The algorithm combines the gene expression levels of the genes UBE2C, KIF20A, PTGER3, OSBPL1A, CYP27A1 and IGKC.

FIG. 3
a. Receiver operating characteristics (ROC) curve of the exemplary algorithm CP2 in 185 ER+/HER2− breast cancer patients treated with anthracycline-containing chemotherapy. The algorithm combines the gene expression levels of the genes UBE2C, KIF20A, PGR, OSBPL1A, CYP27A1 and IGKC. Area under the curves (AUC) is indicated.
b. Kaplan-Meier plot of distant recurrence according to exemplary algorithm CP2 in 185 ER+/HER2− breast cancer patients treated with anthracycline-containing chemotherapy. The algorithm combines the gene expression levels of the genes UBE2C, KIF20A, PGR, OSBPL1A, CYP27A1 and IGKC.

FIG. 4
a. Receiver operating characteristics (ROC) curve of the exemplary algorithm CP2 in 295 ER+/HER2− breast cancer patients treated with taxane/anthracycline-containing chemotherapy. The algorithm combines the gene expression levels of the genes UBE2C, KIF20A, PGR, OSBPL1A, CYP27A1 and IGKC. Area under the curves (AUC) is indicated.
b. Kaplan-Meier plot of distant recurrence according to the predictive exemplary algorithm CP2 in 295 ER+/HER2− breast cancer patients treated with taxane/anthracycline-containing chemotherapy. The algorithm CP2 combines the gene expression levels of the genes UBE2C, KIF20A, PGR, OSBPL1A, CYP27A1 and IGKC.

FIG. 5
a. Receiver operating characteristics (ROC) curve of the exemplary algorithm CPclin1 in 185 ER+/HER2− breast cancer patients treated with anthracycline-containing chemotherapy. The algorithm combines the gene expression levels of the genes UBE2C, KIF20A, PTGER3, OSBPL1A, CYP27A1 and IGKC. Area under the curve (AUC) is indicated.
b. Kaplan-Meier plot of distant recurrence according to the exemplary algorithm CPclin1 in 185 ER+/HER2− breast cancer patients treated with anthracycline-containing chemotherapy. The algorithm combines the gene expression levels of the genes UBE2C, KIF20A, PTGER3, OSBPL1A, CYP27A1 and IGKC.

FIG. 6
a. Receiver operating characteristics (ROC) curve for the of the exemplary algorithm CPclin1 in 295 ER+/HER2− breast cancer patients treated with taxane/anthracycline-containing chemotherapy. The algorithm combines the gene expression levels of the genes UBE2C, KIF20A, PTGER3, OSBPL1A, CYP27A1 and IGKC. Area under the curves (AUC) is indicated.
b. Kaplan-Meier plot of distant recurrence according to the of the exemplary algorithm CPclin1 in 295 ER+/HER2− breast cancer patients treated with taxane/anthracycline-containing chemotherapy. The algorithm combines the gene expression levels of the genes UBE2C, KIF20A, PTGER3, OSBPL1A, CYP27A1 and IGKC.

FIG. 7
a. Receiver operating characteristics (ROC) curve of the exemplary algorithm CPclin2 in 185 ER+/HER2− breast cancer patients treated with anthracycline-containing chemotherapy. The algorithm combines the gene expression levels of the genes UBE2C, KIF20A, PGR, OSBPL1A, CYP27A1 and IGKC. Area under the curves (AUC) is indicated.
b. Kaplan-Meier plot of distant recurrence according to exemplary algorithm CPclin2 in 185 ER+/HER2− breast cancer patients treated with anthracycline-containing chemotherapy. The algorithm combines the gene expression levels of the genes UBE2C, KIF20A, PGR, OSBPL1A, CYP27A1 and IGKC.

FIG. 8
a. Receiver operating characteristics (ROC) curve of the exemplary algorithm CPclin2 in 295 ER+/HER2− breast cancer patients treated with taxane/anthracycline-containing chemotherapy. The algorithm combines the gene expression levels of the genes UBE2C, KIF20A, PGR, OSBPL1A, CYP27A1 and IGKC. Area under the curves (AUC) is indicated.

b. Kaplan-Meier plot of distant recurrence according to the predictive exemplary algorithm CPclin2 in 295 ER+/HER2− breast cancer patients treated with taxane/anthracycline-containing chemotherapy. The algorithm CP2 combines the gene expression levels of the genes UBE2C, KIF20A, PGR, OSBPL1A, CYP27A1 and IGKC.

FIG. 9

Modeled probability of distant metastasis events depending on the exemplary taxane metagene (TM) score: S100P, PCSK6 and STC1.

Red curve=295 ER+/HER2− breast cancer patients treated with taxane/anthracycline-based therapy. Blue curve=185 ER+/HER2− breast cancer patients treated with anthracycline-based therapy.

Tumor samples with a high taxane metagene score have a considerably good outcome when treated with taxan/anthracycline-containing chemotherapy in comparison to anthracycline-based treatment. Test for treatment interaction showed that the taxane metagene score was significantly (p=0.00142237) associated with taxane efficacy.

FIG. 10

Modeled probability of distant metastasis events depending on the predictive score based on the gene expression information of S100P/PCSK6/GPRC5A.

Red curve=295 ER+/HER2− breast cancer patients treated with taxane/anthracycline-based therapy. Blue curve=185 ER+/HER2− breast cancer patients treated with anthracycline-based therapy.

Tumor samples with a high taxane metagene score have a considerably good outcome when treated with taxan/anthracycline-containing chemotherapy in comparison to anthracycline-based treatment. Test for treatment interaction showed that the taxane metagene score was significantly (p=0.040552957) associated with taxane efficacy.

FIG. 11

Modeled probability of distant metastasis events depending on the predictive score based on the gene expression information of S100P/GPRC5A/STC1.

Red curve=295 ER+/HER2− breast cancer patients treated with taxane/anthracycline-based therapy. Blue curve=185 ER+/HER2− breast cancer patients treated with anthracycline-based therapy.

Tumor samples with a high taxane metagene score have a considerably good outcome when treated with taxan/anthracycline-containing chemotherapy in comparison to anthracycline-based treatment. Test for treatment interaction showed that the taxane metagene score was significantly (p=0.005130854) associated with taxane efficacy.

FIG. 12

Modeled probability of distant metastasis events depending on the predictive score based on the gene expression information of PCSK6/GPRC5A/STC1.

Red curve=295 ER+/HER2− breast cancer patients treated with taxane/anthracycline-based therapy. Blue curve=185 ER+/HER2− breast cancer patients treated with anthracycline-based therapy.

Tumor samples with a high taxane metagene score have a considerably good outcome when treated with taxan/anthracycline-containing chemotherapy in comparison to anthracycline-based treatment. Test for treatment interaction showed that the taxane metagene score was significantly (p=0.00049102) associated with taxane efficacy.

FIG. 13

Modeled probability of distant metastasis events depending on the predictive score based on the gene expression information of S100P/STC1.

Red curve=295 ER+/HER2− breast cancer patients treated with taxane/anthracycline-based therapy. Blue curve=185 ER+/HER2− breast cancer patients treated with anthracycline-based therapy.

Tumor samples with a high taxane metagene score have a considerably good outcome when treated with taxan/anthracycline-containing chemotherapy in comparison to anthracycline-based treatment. Test for treatment interaction showed that the taxane metagene score was significantly (p=0.008397803) associated with taxane efficacy.

FIG. 14

Modeled probability of distant metastasis events depending on the predictive score based on the gene expression information of PCSK6/STC1.

Red curve=295 ER+/HER2− breast cancer patients treated with taxane/anthracycline-based therapy. Blue curve=185 ER+/HER2− breast cancer patients treated with anthracycline-based therapy.

Tumor samples with a high taxane metagene score have a considerably good outcome when treated with taxan/anthracycline-containing chemotherapy in comparison to anthracycline-based treatment. Test for treatment interaction showed that the taxane metagene score was significantly (p=0.000914024) associated with taxane efficacy.

FIG. 15

Modeled probability of distant metastasis events depending on the predictive score based on the gene expression information of GPRC5A/PCSK6.

Red curve=295 ER+/HER2− breast cancer patients treated with taxane/anthracycline-based therapy. Blue curve=185 ER+/HER2− breast cancer patients treated with anthracycline-based therapy.

Tumor samples with a high taxane metagene score have a considerably good outcome when treated with taxan/anthracycline-containing chemotherapy in comparison to anthracycline-based treatment. Test for treatment interaction showed that the taxane metagene score was significantly (p=0.04090109) associated with taxane efficacy.

FIG. 16

Modeled probability of distant metastasis events depending on the predictive score based on the gene expression information of S100P/GPRC5A.

Red curve=295 ER+/HER2− breast cancer patients treated with taxane/anthracycline-based therapy. Blue curve=185 ER+/HER2− breast cancer patients treated with anthracycline-based therapy.

Tumor samples with a high taxane metagene score have a considerably good outcome when treated with taxan/anthracycline-containing chemotherapy in comparison to anthracycline-based treatment. Test for treatment interaction showed that the taxane metagene score was significantly (p=0.11173176) associated with taxane efficacy.

FIG. 17

Modeled probability of distant metastasis events depending on the predictive score based on the gene expression information of GPRC5A/STC1.

Red curve=295 ER+/HER2− breast cancer patients treated with taxane/anthracycline-based therapy. Blue curve=185 ER+/HER2− breast cancer patients treated with anthracycline-based therapy.

Tumor samples with a high taxane metagene score have a considerably good outcome when treated with taxan/anthracycline-containing chemotherapy in comparison to anthracycline-based treatment. Test for treatment interaction showed that the taxane metagene score was significantly (p=0.00260675) associated with taxane efficacy.

FIG. 18

Platform transfer—PTGER3: The results from the Affymetrix data (log2 expression data) in fresh-frozen tumor samples were transferred to a diagnostic platform (qRT-PCR, dCt level) and formalin-fixed paraffin-embedded tissue using 56 paired technical samples.

### DETAILED DESCRIPTION OF THE INVENTION

Additional details, features, characteristics and advantages of the object of the invention are disclosed in the sub-claims, and the following description of the respective figures, tables and examples, which, in an exemplary fashion, show preferred embodiments of the present invention. However, these drawings should by no means be understood as to limit the scope of the invention.

Two gene expression data sets (n=480; Affymetrix HG-U133A) were used to establish predictive algorithms. All analyzed breast cancer patients were treated with anthracycline or taxan/anthracycline-containing chemotherapy. Micro-array cell files were MAS5 normalized with a global scaling procedure and a target intensity of 500. The analysis was performed in ER-positive, HER2− negative breast cancer patients according to pre-specified cut-off levels (ERBB2 probeset 216836<6000 and ESR1 probeset >1000=ER-positive/HER2-negative).

Several marker genes were identified that predicted the residual risk of recurrence after standard chemotherapy in both datasets (Tables 1, 2). Primer and probe sequences for the marker genes are shown in table 3. Based on the expression values of the predictive marker genes, combined scores were calculated by a mathematical combination, e.g. a linear combination. Two exemplary algorithms CP1 and CP2 (prognostic combined scores consisting of six genes of interest) were established and coefficients were determined by multi-variate COX regression. It was found that the prognostic combined CP1 score (containing six genes of interest: KIF20A, UBE2C, PTGER3, OSBPL1A, IGKC, CYP27A1) was particular suited for predicting the residual risk of recurrence after standard chemotherapy treatment (FIG. 1/2). A high score indicates a high risk of developing metastases after standard chemotherapy treatment, whereas a low CP1 score indicates a decreased likelihood (FIG. 1/2).

Several other combinations of candidate genes (listed in table 1/2) were also found to predict risk of recurrence after standard chemotherapy treatment (table 4). The combined CP2 score (genes of interest: KIF20A, UBE2C, PGR, OSBPL1A, IGKC, CYP27A1) was particularly valuable to identify patients with low or high probability of survival following standard chemotherapy (FIG. 3, 4).

The molecular scores (table 4) were combined with the clinical information: nodal status. The hybrid scores showed an improved classification performance compared to the molecular scores alone. CP1clin (CP1 score+nodal status) and CP2clin (CP2 score+nodal status) were particularly valuable to identify patients with low or high probability of survival following standard chemotherapy (FIG. 5-8).

Additionally, the methods of the invention are suited to predict the benefit from inclusion of taxane in a chemotherapy regimen in breast cancer patients.

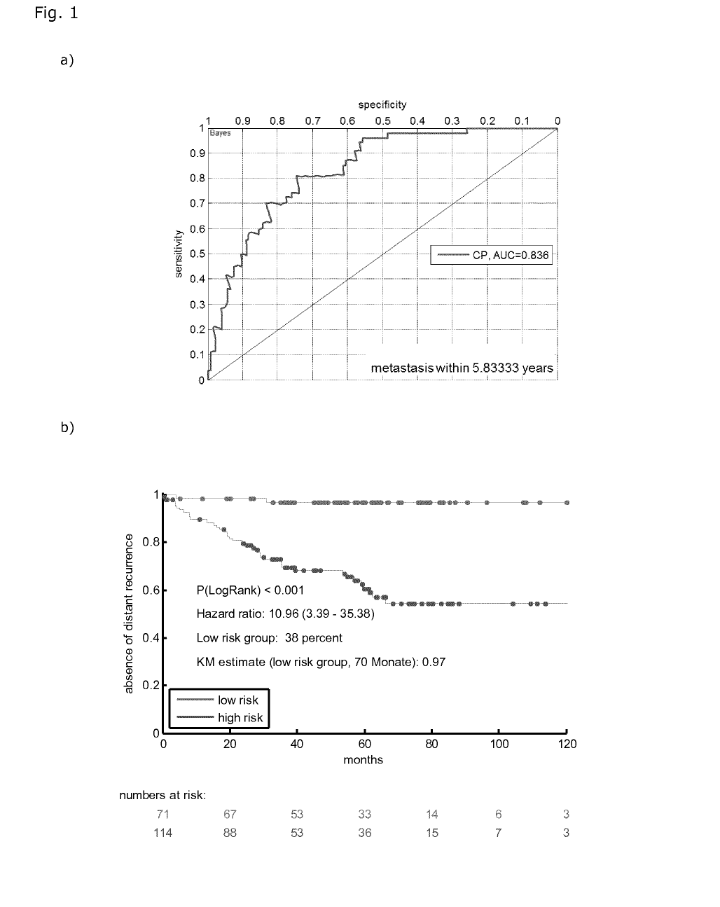

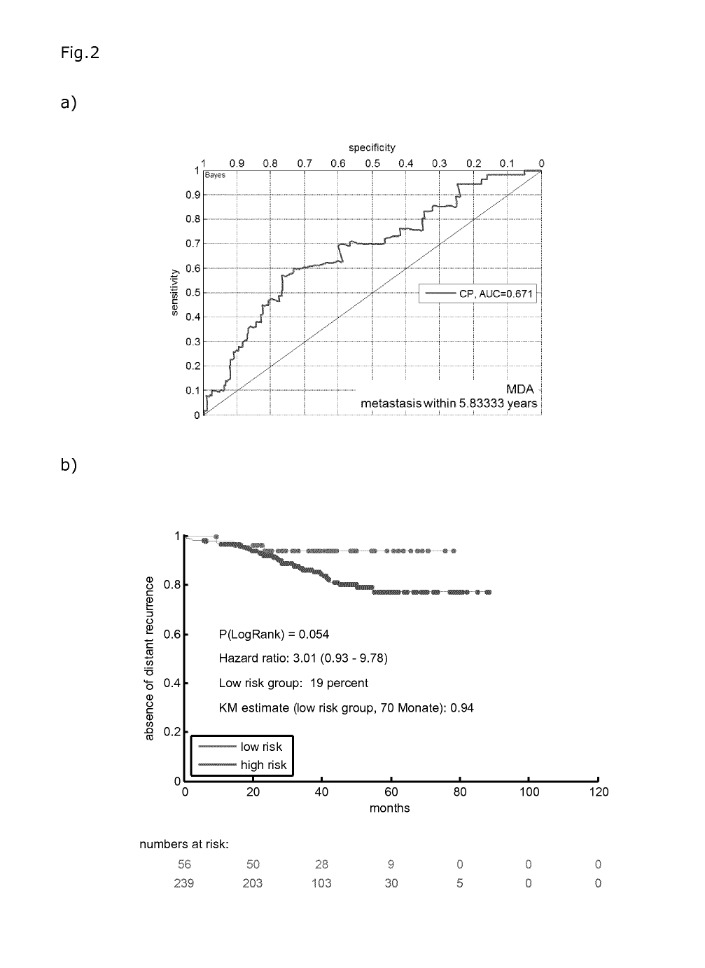

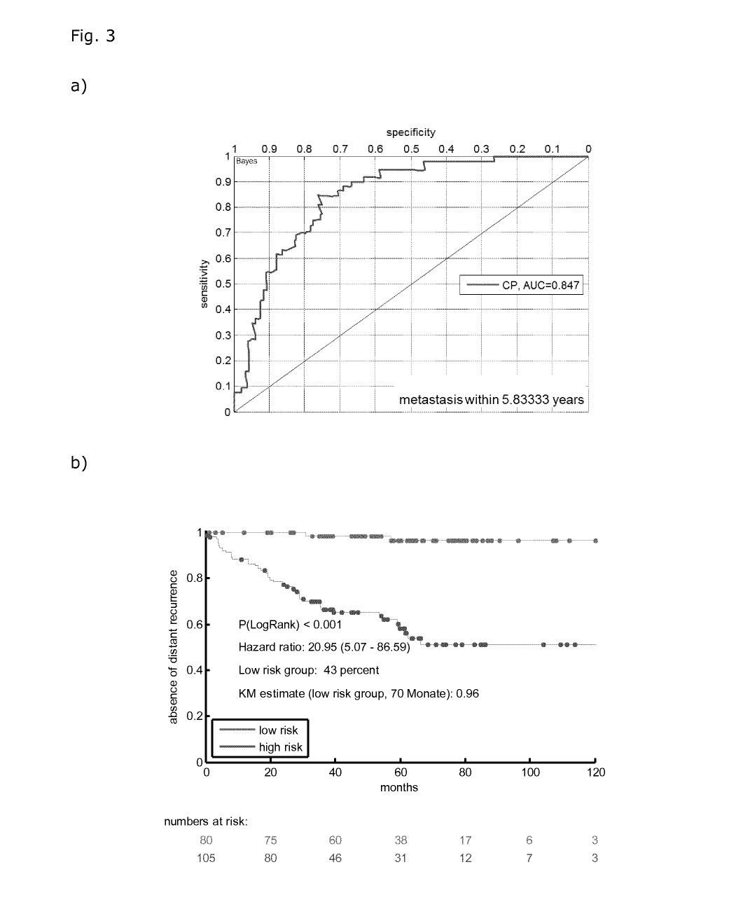

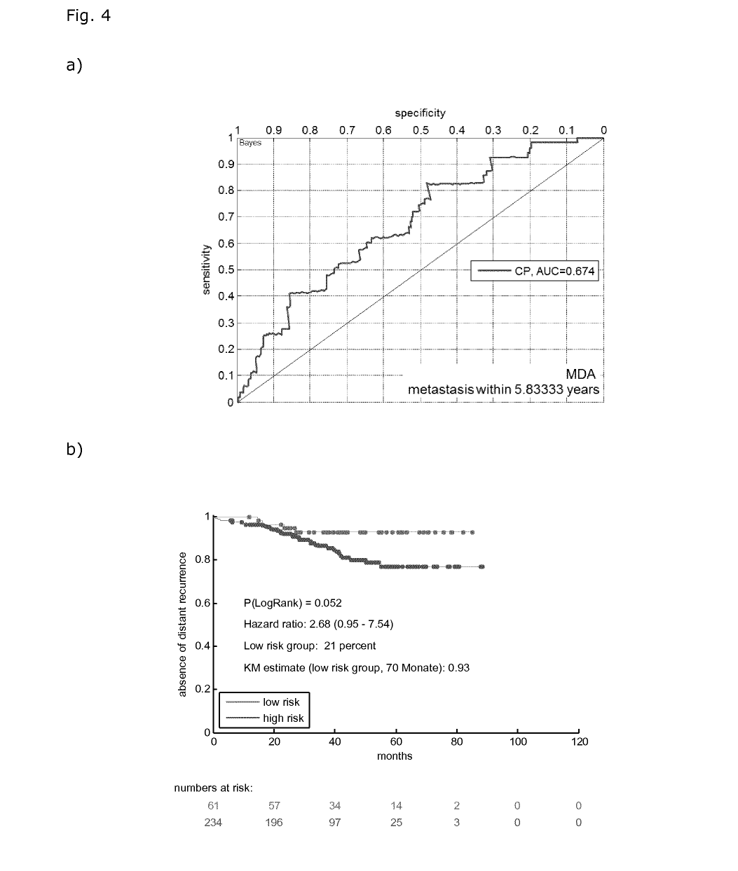

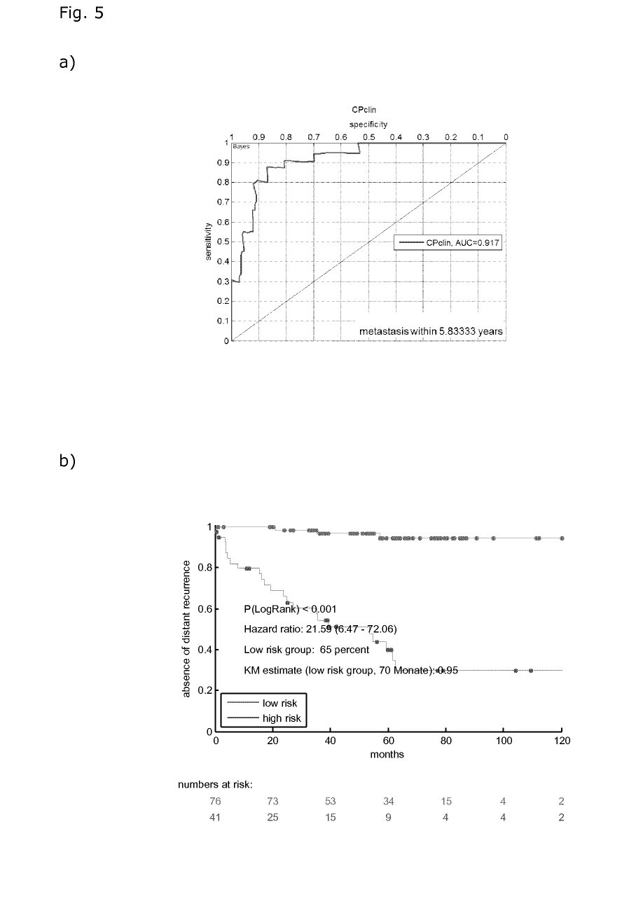

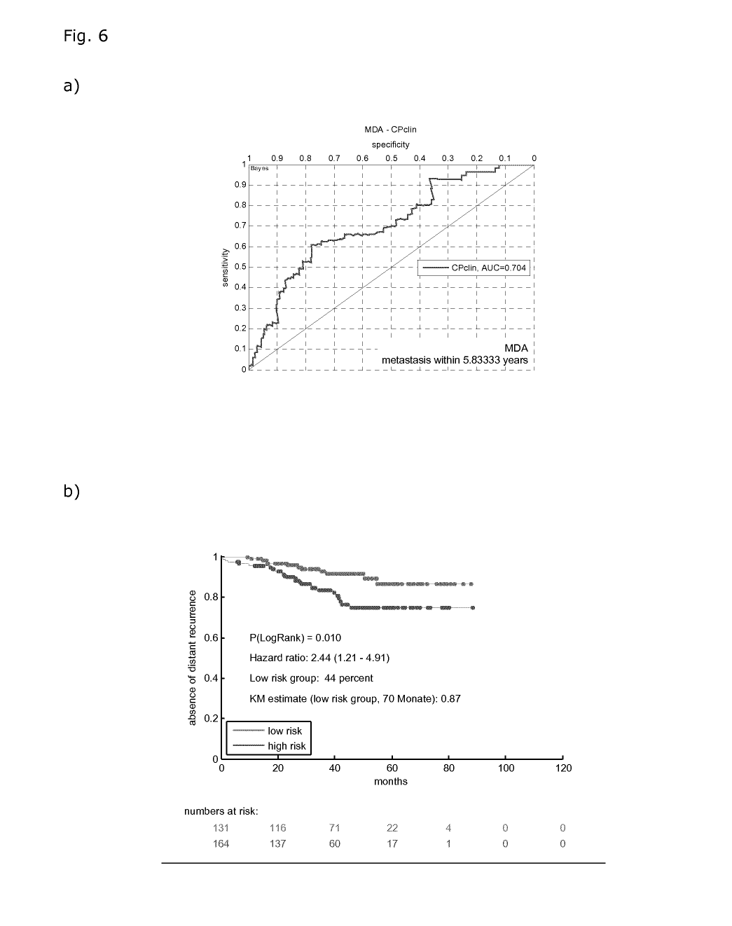

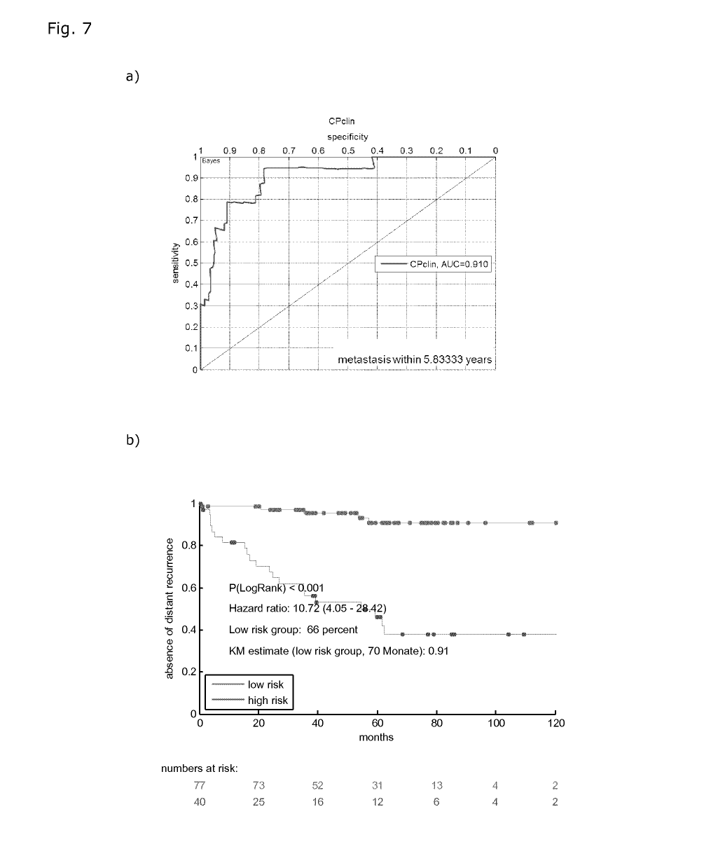

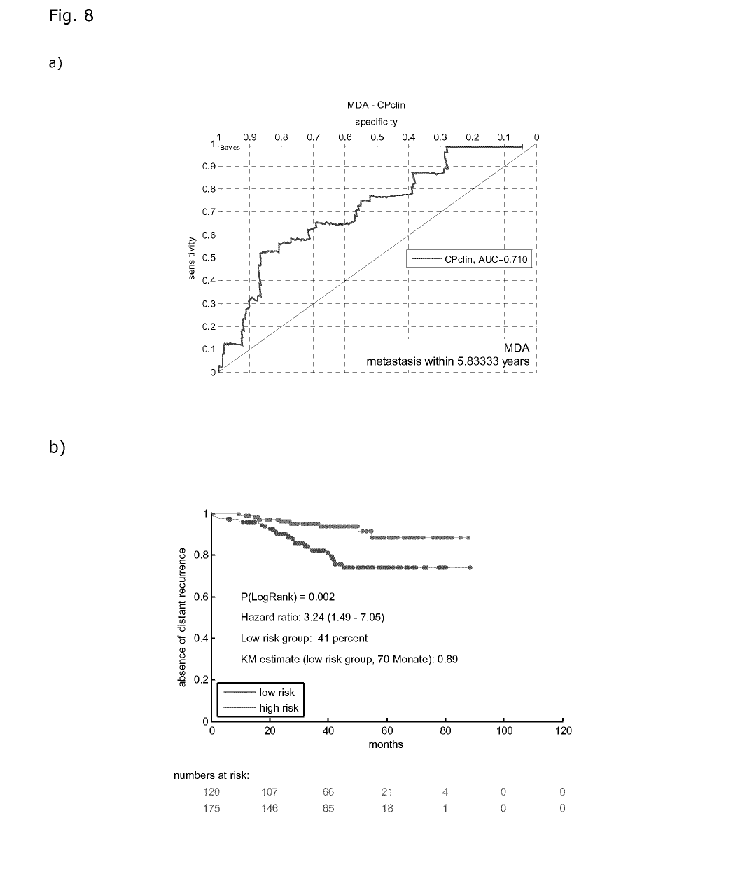

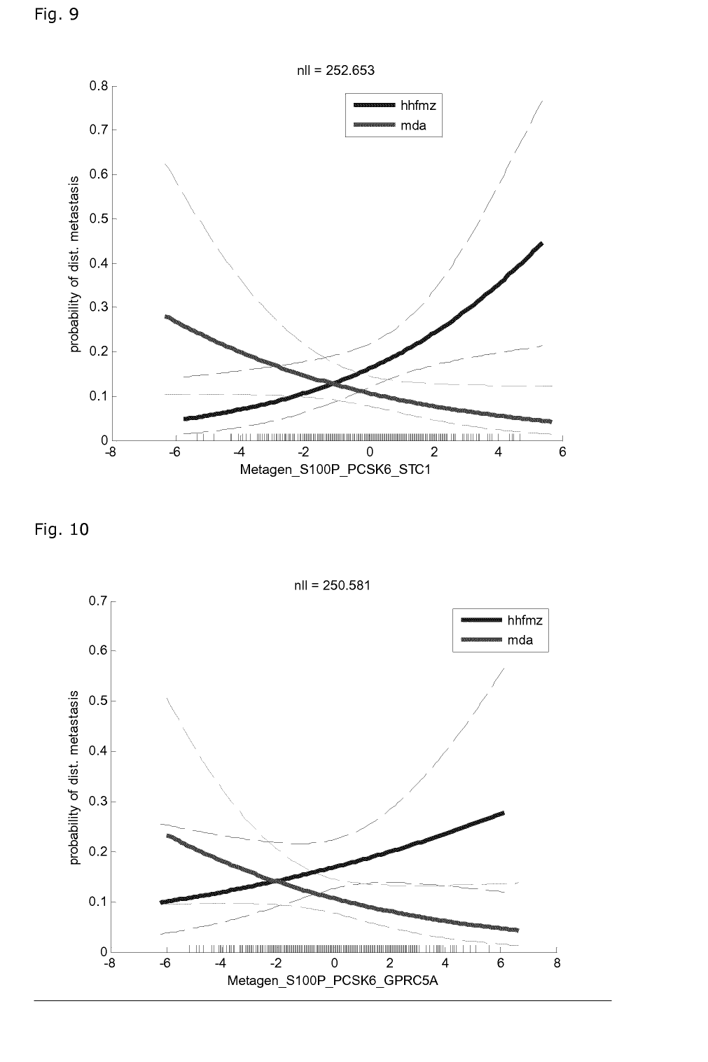

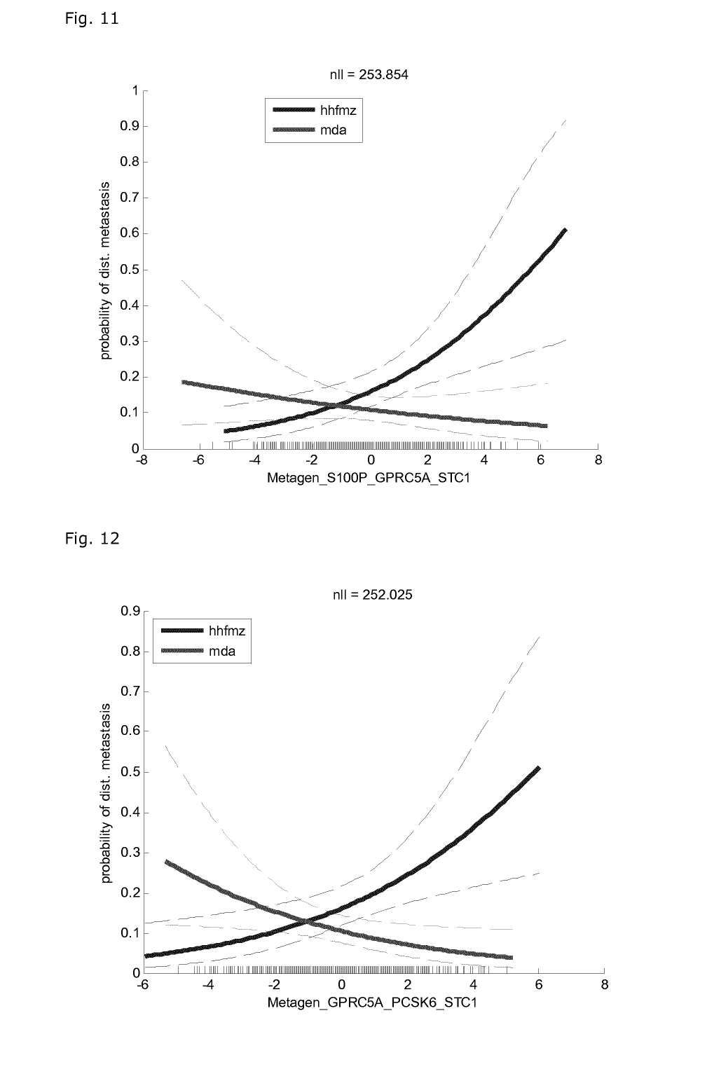

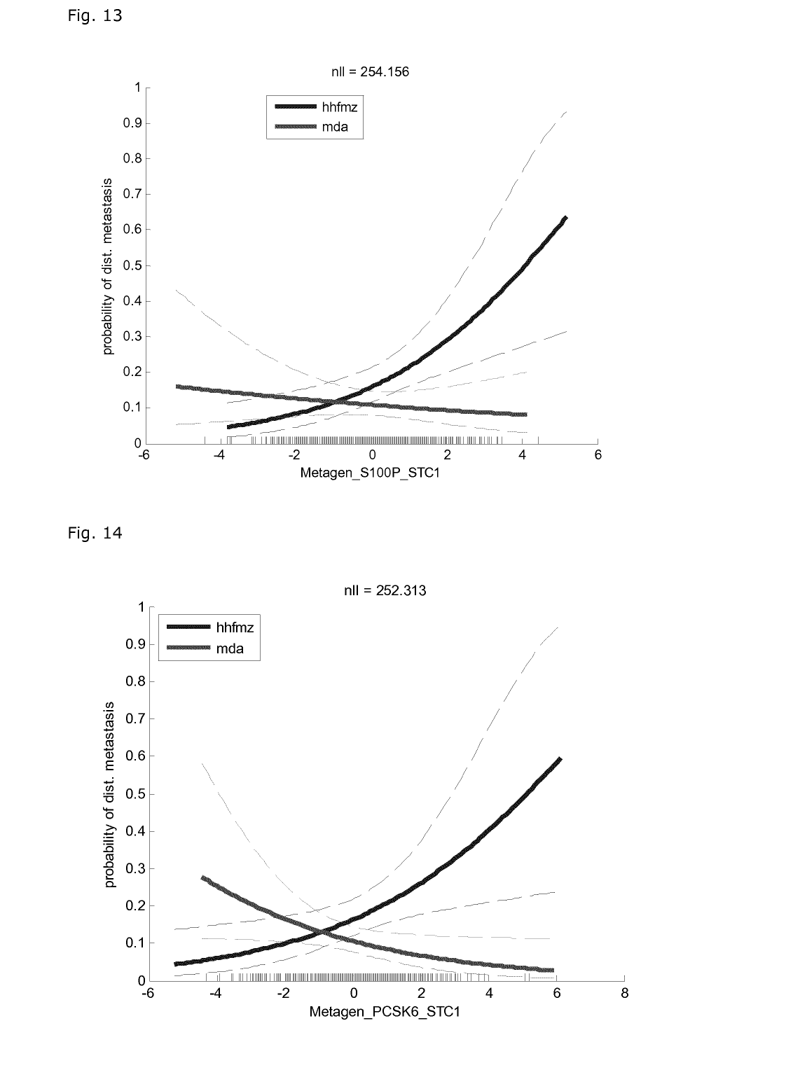

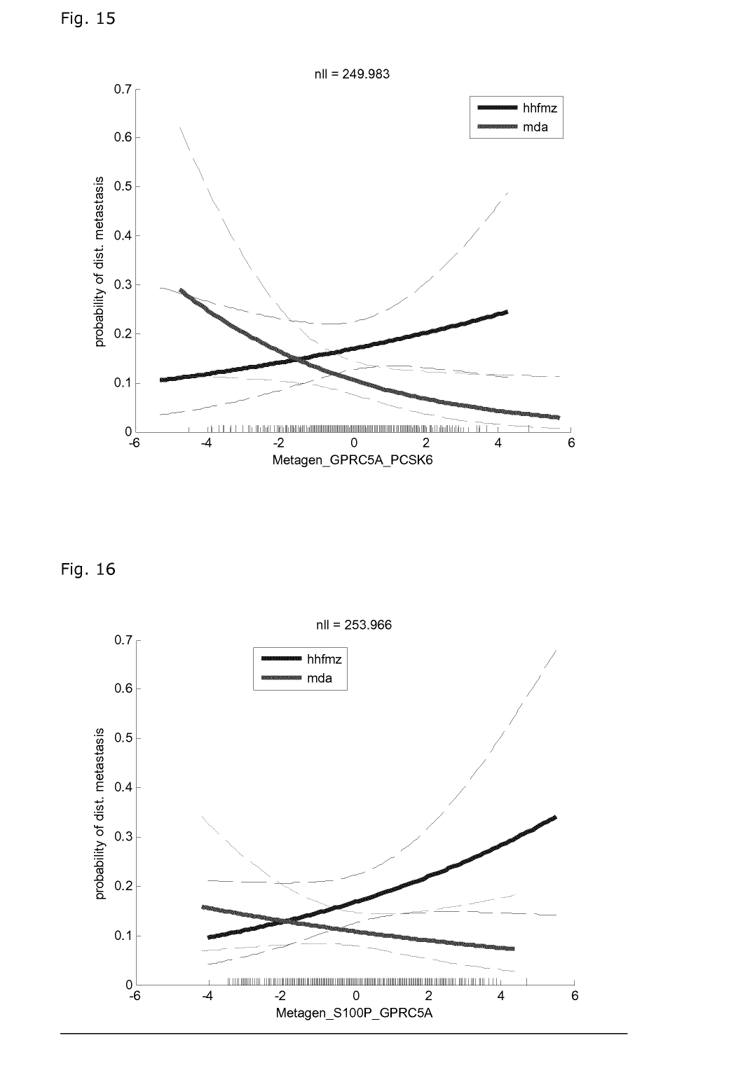

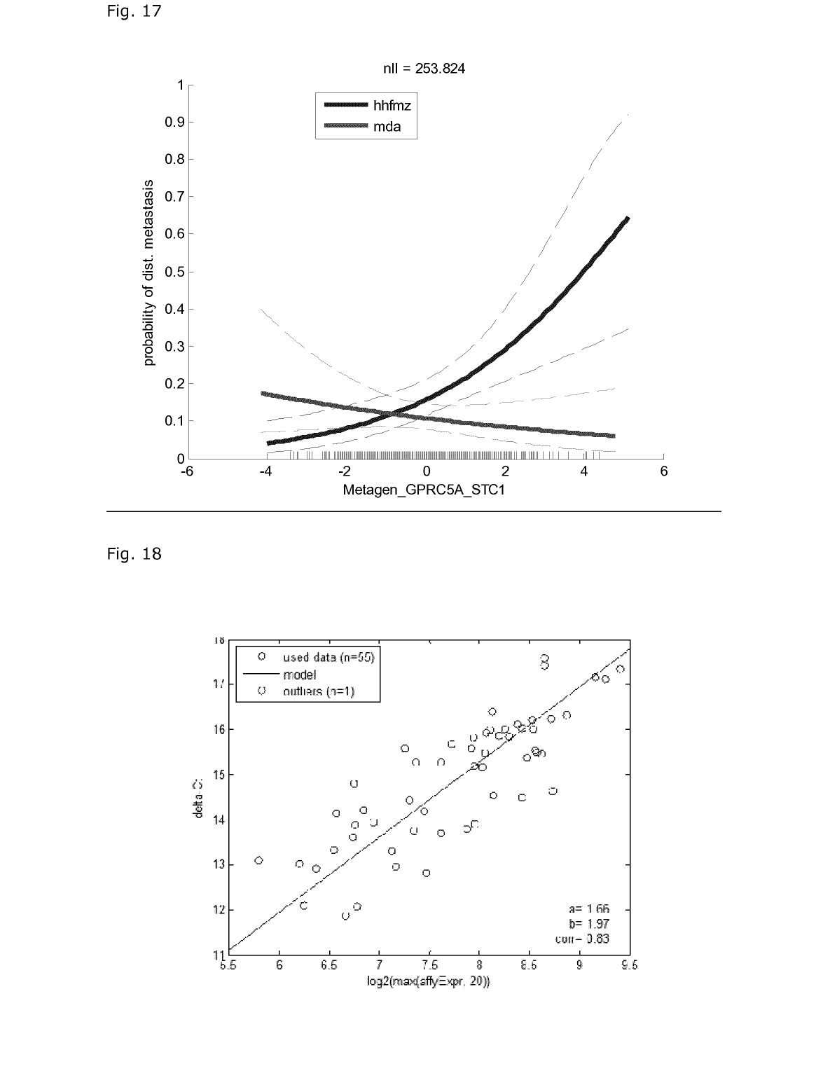

It was found that the gene expression levels of S100P, STC1, PCSK6 and GPRC5A are generally indicative for benefit from taxane-containing therapy in breast cancer. A high expression level of the four genes indicates an increased likelihood of benefit from inclusion of taxane in a chemotherapy regimen. The combination of the three genes S100P, STC1, PCSK6 has been found to be particularly valuable to predict taxane efficacy (FIG. 9). A high score indicates an increased likelihood of a benefit from inclusion of taxane in a chemotherapy regimen (FIG. 9), whereas the subcohort with a low score has no benefit or even an adverse effect regarding outcome. Several other combinations of the predictive marker genes also allowed predicting taxane efficacy (FIGS. 10-17).

Herein disclosed are unique combinations of marker genes which can be combined into an algorithm for the here presented new predictive test. Technically, the method of the invention can be practiced using two technologies: 1.) Isolation of total RNA from fresh or fixed tumor tissue and 2.) Quantitative RT-PCR of the isolated nucleic acids. Alternatively, it is known to everybody skilled in the art that expression levels can also be measured using alternative technologies, including but not limited to microarray, in particular Affymetrix U-133A arrays, sequencing or by measurement at a protein level.

The methods of the invention are based on quantitative determination of RNA species isolated from the tumor in order to obtain expression values and subsequent bioinformatics analysis of said determined expression values. RNA species can be isolated from any type of tumor sample, e.g. biopsy samples, smear samples, resected tumor material, fresh frozen tumor tissue or from paraffin embedded and formalin fixed tumor tissue.

The results from the Affymetrix data in fresh-frozen tumor samples were transferred to a diagnostic platform (qRT-PCR) and formalin-fixed paraffin-embedded tissue using 56 paired technical samples. The platform transfer was done using Affymetrix microarray data (fresh-frozen tumor samples) and qRT-PCR expression data (FFPE samples) from the same technical samples (example—FIG. 18).

Herein disclosed is a unique panel of genes which can be combined into algorithms for the here presented new predictive test.

TABLE 1
Affymetrix probeset ID and TaqMan design ID mapping of
the marker genes of the present invention.

| Gene | Design ID | Probeset ID |
|---|---|---|
| S100P | SVD0018 | 204351_at |
| PCSK6 | SVD0016 | 207414_s_at |
| STC1 | CAGMC424 | 204595_s_at |
| PTGER3 | CAGMC315 | 210832_x_at |
| OSBPL1A | SVD0050 | 208158_s_at |
| KIF20A | SVD0020 | 218755_at |
| UBE2C | R65 | 202954_at |
| IGKC | R61 | 211645_x_at |
| CYP27A1 | SVD0003 | 203979_at |
| PGR | BC172 | 208305_at |
| CHPT1 | R138 | 221675_s_at |
| RACGAP1 | R125-2 | 222077_s_at |
| TOP2A | R70 | 201292_at |
| AURKA | CAGMC336 | 204092_s_at |
| GPRC5A | SVD0049 | 203108_at |
| CXCL13 | R109 | 205242_at |
| CCL5 | SVD0015 | 1405_i_at |

TABLE 2
Gene names, Entrez Gene ID and chromosomal location of the
marker genes of the present invention

| Official | | | |
|---|---|---|---|
| Symbol | Official Full Name | Entrez¹ | Location |
| S100P | S100 calcium binding protein P | 6286 | 4p16 |
| PCSK6 | proprotein convertase subtilisin/ | 5046 | 15q26.3 |
| | kexin type 6 | | |
| STC1 | stanniocalcin 1 | 6781 | 8p21-p11.2 |
| PTGER3 | prostaglandin E receptor 3 (subtype | 5733 | 1p31.2 |
| | EP3) | | |
| OSBPL1A | oxysterol binding protein-like 1A | 114876 | 18q11.1 |
| KIF20A | kinesin family member 20A | 10112 | 5q31 |
| UBE2C | ubiquitin-conjugating enzyme E2C | 11065 | 20q13.12 |
| IGKC | immunoglobulin kappa constant | 3514 | 2p12 |
| CYP27A1 | cytochrome P450, family 27, | 1593 | 2q35 |
| | subfamily A, polypeptide 1 | | |
| PGR | progesterone receptor | 5241 | 11q22-q23 |
| CHPT1 | choline phosphotransferase 1 | 56994 | 12q |
| RACGAP1 | Rac GTPase activating protein 1 | 29127 | 12q13.12 |
| TOP2A | topoisomerase (DNA) II alpha | 7153 | 17q21-q22 |
| | 170 kDa | | |
| AURKA | aurora kinase A | 6790 | 20q13 |
| GPRC5A | G protein-coupled receptor, family | 9052 | 12p13-p12.3 |
| | C, group 5, member A | | |
| CXCL13 | chemokine (C-X-C motif) ligand 13 | 10563 | 4q21 |
| CCL5 | chemokine (C-C motif) ligand 5 | 6352 | 17q12 |

¹Entrez Gene Identification is linked to the data base NCBI: http://www.ncbi.nlm.nih.gov/gene

TABLE 3
Primer and probe sequences

| SEQ ID | | | |
|---|---|---|---|
| NO: | Gene symbol | Primer-ID | Probe |
| 1 | S100P | SVD0018 | CTGCAATCACGTCTGCCTGTCACAAGT |
| 4 | PCSK6 | SVD0016 | CTGCTCCCCTGTTTGACGACAGTGC |
| 7 | STC1 | CAGMC424 | CCTTTCATCGGTGCCTGGTACTCTGG |
| 10 | PTGER3 | CAGMC315 | TCGGTCTGCTGGTCTCCGCTCC |
| 13 | OSBPL1A | SVD0050 | CTCCACCCGCCAGCATCCTTAGC |
| 16 | KIF20A | SVD0020 | TCCCCGAACACCAACCTGCCA |
| 19 | UBE2C | R65 | TGAACACACATGCTGCCGAGCTCTG |
| 28 | IGKC | R61 | AGCAGCCTGCAGCCTGAAGATTTTGC |
| 22 | CYP27A1 | SVD0003 | AAACAGCCAGCCTGCTACCCCCAG |
| 25 | PGR | BC172 | TTGATAGAAACGCTGTGAGCTCGA |
| 31 | CHPT1 | R138 | CCACGGCCACCGAAGAGGCAC |
| 34 | RACGAP1 | R125-2 | ACTGAGAATCTCCACCCGGCGCA |
| 37 | TOP2A | R70 | CAGATCAGGACCAAGATGGTTCCCACAT |
| 40 | AURKA | CAGMC336 | CCGTCAGCCTGTGCTAGGCAT |
| 43 | GPRC5A | SVD0049 | ATCTCCCCCTACGCTCTGCCAGGA |
| 46 | CXCL13 | R109 | TGGTCAGCAGCCTCTCTCCAGTCCA |
| 49 | CCL5 | SVD0015 | CTCTGCGCTCCTGCATCTGCCTC |
| SEQ ID | | | |
| NO: | Gene symbol | Primer-ID | Forward Primer |
| 2 | S100P | SVD0018 | TTCAGTGAGTTCATCGTGTTCGT |
| 5 | PCSK6 | SVD0016 | TTTCGACCTCGTCTTTCTCCAT |
| 8 | STC1 | CAGMC424 | ACATTAGGAAGTGGCAGTTCTTTACTC |
| 11 | PTGER3 | CAGMC315 | CTGATTGAAGATCATTTTCAACATCA |
| 14 | OSBPL1A | SVD0050 | TGAATTAGAGCAGTCTCTGGTGAAAG |
| 17 | KIF20A | SVD0020 | AACCACCAGGGAAGAAACCATT |
| 20 | UBE2C | R65 | CTTCTAGGAGAACCCAACATTGATAGT |
| 29 | IGKC | R61 | GATCTGGGACAGAATTCACTCTCA |
| 23 | CYP27A1 | SVD0003 | CTGCCTTCTCTGAGCCTGAAA |
| 26 | PGR | BC172 | AGCTCATCAAGGCAATTGGTTT |
| 32 | CHPT1 | R138 | CGCTCGTGCTCATCTCCTACT |
| 35 | RACGAP1 | R125-2 | TCGCCAACTGGATAAATTGGA |
| 38 | TOP2A | R70 | CATTGAAGACGCTTCGTTATGG |
| 41 | AURKA | CAGMC336 | AATCTGGAGGCAAGGTTCGA |
| 44 | GPRC5A | SVD0049 | ACTTGCTGTCAATTCCGAGATCT |
| 47 | CXCL13 | R109 | CGACATCTCTGCTTCTCATGCT |
| 50 | CCL5 | SVD0015 | CGCTGTCATCCTCATTGCTACT |
| SEQ ID | | | |
| NO: | Gene symbol | Primer-ID | Reverse Primer |
| 3 | S100P | SVD0018 | CATCATTTGAGTCCTGCCTTCTC |
| 6 | PCSK6 | SVD0016 | TCTCTCCAGCTCACAGGTGACA |
| 9 | STC1 | CAGMC424 | CTCCCACCCCATCATCATTT |
| 12 | PTGER3 | CAGMC315 | GACGGCCATTCAGCTTATGG |
| 15 | OSBPL1A | SVD0050 | GAATCTGACAGCGCATCATAGAA |
| 18 | KIF20A | SVD0020 | GCATAAGGGCTGCAGTCTGTT |
| 21 | UBE2C | R65 | GTTTCTTGCAGGTACTTCTTAAAAGCT |
| 30 | IGKC | R61 | GCCGAACGTCCAAGGGTAA |
| 24 | CYP27A1 | SVD0003 | CAAAGGGCACAGAGCCAAA |
| 27 | PGR | BC172 | ACAAGATCATGCAAGTTATCAAGAAGTT |
| 33 | CHPT1 | R138 | CCCAGTGCACATAAAAGGTATGTC |
| 36 | RACGAP1 | R125-2 | GAATGTGCGGAATCTGTTTGAG |
| 39 | TOP2A | R70 | CCAGTTGTGATGGATAAAATTAATCAG |
| 42 | AURKA | CAGMC336 | TCTGGATTTGCCTCCTGTGAA |
| 45 | GPRC5A | SVD0049 | GGCTTGTGCTAGTGAGGTCTGA |
| 48 | CXCL13 | R109 | AGCTTGTGTAATAGACCTCCAGAACA |
| 51 | CCL5 | SVD0015 | TGTGGTGTCCGAGGAATATGG |

### EXAMPLE 1

### Algorithm CP1

The CP1 algorithm is a linear combination of the expression levels of UBE2C, OSBPL1A, IGKC, KIF20A, PTGER3 and CYP27A1. The mathematical formulas for CP1 are shown below; the score can be calculated from gene expression data. Relative expression levels of genes of interest (GOI) can be calculated as ΔCt values (ΔCt=20−[CtGOI−Ct(mean of RPL37A, CALM2, OAZ1)]).

CP1=0.418839ΔCₜ(UBE2C)−0.270581ΔCₜ(OSBPL1A)−0.160038ΔCₜ(IGKC) +0.612913+0.466064ΔCₜ(KIF20A)−0.191108ΔCₜ(PTGER3)−0.389215ΔCₜ(CYP27A1)+1.973329

### CP1clin

In a preferred embodiment CP1clin is a combined score consisting of the CP1 algorithm (see above) and nodal status.

CP1clin=0.544508CP1+0.564300 nodal status

where nodal status (1: negative, 2: 1 to 3 positive nodes, 3: 4 to 9 positive nodes, 4: >9 positive nodes).

### EXAMPLE 2

### Algorithm CP2

The CP2 algorithm is a linear combination of the expression levels of UBE2C, OSBPL1A, IGKC, KIF20A, PGR and CYP27A1. The mathematical formulas for CP1 are shown below; the score can be calculated from gene expression data only. Relative expression levels of genes of interest (GOI) can be calculated as ΔCt values (ΔCt=20−[CtGOI−Ct(mean of RPL37A, CALM2, OAZ1)]).

CP2=0.418839ΔCₜ(UBE2C)−0.270581ΔCₜ(OSBPL1A)−0.160038ΔCₜ(IGKC) +0.612913+0.492345ΔCₜ(KIF20A)−0.138801ΔCₜ(PGR)−0.371736ΔCₜ(CYP27A1)+0.644467

### CP2clin

In a preferred in embodiment CP2clin is a combined score consisting of the CP2 algorithm and nodal status.

CP2clin=0.542765CP2+0.568982 nodal status

where nodal status (1: negative, 2: 1 to 3 positive nodes, 3: 4 to 9 positive nodes, 4: >9 positive nodes).

### EXAMPLE

### TM Algorithm

The TM algorithm is a linear score predicting taxane efficacy in breast cancer. Relative expression levels of genes of interest (GOI) can be calculated as ΔCt values (ΔCt=20−[CtGOI−Ct(mean of RPL37A, CALM2, OAZ1)]).

Taxane Metagene=0.665399ΔCₜ(S100P)+0.818044ΔCₜ(PCSK6)+0.606981ΔCₜ(STC1)−30.199475

TaxoClin=0.173176 Taxane Metagene−0.212030 grading

where grading (0: G1 and G2, 1: G3).

TABLE 4
Prognostic multigene scores for predicting residual risk of recurrence
after standard chemotherapy treatment.
The c-index and the area under the ROC curve (AUC) were used to assess the
prognostic performance of the different signatures. A c-index or AUC of 0.5
indicates that the combined score has no prognostic information, whereas in-
creased c-index or AUC values (>0.5) are associated with an improved
prognostic performance.

| Score | c-index | AUC |
|---|---|---|
| Score1 = 0.418839 ΔCₜ(UBE2C) − 0.270581 | 0.78481223 | 0.83578165 |
| ΔCₜ(OSBPL1A) − 0.160038 ΔCₜ(IGKC) + 0.612913 + | | |
| 0.466064 ΔCₜ(KIF20A) − 0.191108 ΔCₜ(PTGER3) − | | |
| 0.389215 ΔCₜ(CYP27A1) + 1.973329 | | |
| Score2 = 0.395246 ΔCₜ(UBE2C) − 0.238484 | 0.78846793 | 0.83965092 |
| ΔCₜ(OSBPL1A) − 0.293624 ΔCₜ(CYP27A1) + | | |
| 2.266142 + 0.468216 ΔCₜ(KIF20A) − 0.185921 | | |
| ΔCₜ(PTGER3) − 0.080326 ΔCₜ(CXCL13) − 2.613357 | | |
| Score3 = 0.395246 ΔCₜ(UBE2C) − 0.238484 | 0.7813227 | 0.82982166 |
| ΔCₜ(OSBPL1A) − 0.293624 ΔCₜ(CYP27A1) + | | |
| 2.266142 + 0.484992 ΔCₜ(KIF20A) − 0.185992 | | |
| ΔCₜ(PTGER3) − 0.101597 ΔCₜ(CCL5) − 2.117392 | | |
| Score4 = 0.418839 ΔCₜ(UBE2C) − 0.270581 | 0.78614158 | 0.84039167 |
| ΔCₜ(OSBPL1A) − 0.160038 ΔCₜ(IGKC) + 0.612913 + | | |
| 0.468216 ΔCₜ(KIF20A) − 0.185921 ΔCₜ(PTGER3) − | | |
| 0.080326 ΔCₜ(CXCL13) − 2.613357 | | |
| Score5 = 0.418839 ΔCₜ(UBE2C) − 0.270581 | 0.77849784 | 0.82606013 |
| ΔCₜ(OSBPL1A) − 0.160038 ΔCₜ(IGKC) + 0.612913 + | | |
| 0.484992 ΔCₜ(KIF20A) − 0.185992 ΔCₜ(PTGER3) − | | |
| 0.101597 ΔCₜ(CCL5) − 2.117392 | | |
| Score6 = 0.386833 ΔCₜ(UBE2C) − 0.127629 | 0.79710867 | 0.8568515 |
| ΔCₜ(PGR) − 0.187999 ΔCₜ(IGKC) − 1.039180 + | | |
| 0.466064 ΔCₜ(KIF20A) − 0.191108 ΔCₜ(PTGER3) − | | |
| 0.389215 ΔCₜ(CYP27A1) + 1.973329 | | |
| Score7 = 0.388145 ΔCₜ(UBE2C) − 0.161500 | 0.78763709 | 0.83088062 |
| ΔCₜ(PTGER3) − 0.176316 ΔCₜ(IGKC) − 0.766954 + | | |
| 0.521167 ΔCₜ(KIF20A) − 0.240843 ΔCₜ(CHPT1) − | | |
| 0.374667 ΔCₜ(CYP27A1) + 2.423669 | | |
| Score8 = 0.418839 ΔCₜ(UBE2C) − 0.270581 | 0.79544699 | 0.84710375 |
| ΔCₜ(OSBPL1A) − 0.160038 ΔCₜ(IGKC) + 0.612913 + | | |
| 0.492345 ΔCₜ(KIF20A) − 0.138801 ΔCₜ(PGR) − | | |
| 0.371736 ΔCₜ(CYP27A1) + 0.644467 | | |
| Score9 = 0.418839 ΔCₜ(UBE2C) − 0.270581 | 0.7818212 | 0.81975038 |
| ΔCₜ(OSBPL1A) − 0.160038 ΔCₜ(IGKC) + 0.612913 + | | |
| 0.521167 ΔCₜ(KIF20A) − 0.240843 ΔCₜ(CHPT1) − | | |
| 0.374667 ΔCₜ(CYP27A1) + 2.423669 | | |
| Score10 = 0.418839 ΔCₜ(UBE2C) − 0.270581 | 0.78248588 | 0.83654778 |
| ΔCₜ(OSBPL1A) − 0.160038 ΔCₜ(IGKC) + 0.612913 + | | |
| 0.444946 ΔCₜ(RΔCGAP1) − 0.194029 | | |
| ΔCₜ(PTGER3) − 0.392396 ΔCₜ(CYP27A1) + | | |
| 2.212664 | | |
| Score11 = 0.418839 ΔCₜ(UBE2C) − 0.270581 | 0.77500831 | 0.82769742 |
| ΔCₜ(OSBPL1A) − 0.160038 ΔCₜ(IGKC) + 0.612913 + | | |
| 0.332343 ΔCₜ(TOP2A) − 0.193924 ΔCₜ(PTGER3) − | | |
| 0.384761 ΔCₜ(CYP27A1) + 3.559894 | | |
| Score12 = 0.418839 ΔCₜ(UBE2C) − 0.270581 | 0.79012961 | 0.84527199 |
| ΔCₜ(OSBPL1A) − 0.160038 ΔCₜ(IGKC) + 0.612913 + | | |
| 0.337748 ΔCₜ(AURKA) − 0.206178 ΔCₜ(PTGER3) − | | |
| 0.368621 ΔCₜ(CYP27A1) + 3.548630 | | |
| Score13 = 0.531530 ΔCₜ(KIF20A) − 0.283226 | 0.78614158 | 0.83050217 |
| ΔCₜ(OSBPL1A) − 0.318978 ΔCₜ(CYP27A1) + | | |
| 1.933827 + 0.447858 ΔCₜ(RΔCGAP1) − 0.199809 | | |
| ΔCₜ(PTGER3) − 0.178444 ΔCₜ(IGKC) − 0.701929 | | |
| Score14 = 0.531530 ΔCₜ(KIF20A) − 0.283226 | 0.777667 | 0.82240361 |
| ΔCₜ(OSBPL1A) − 0.318978 ΔCₜ(CYP27A1) + | | |
| 1.933827 + 0.327216 ΔCₜ(TOP2A) − 0.197938 | | |
| ΔCₜ(PTGER3) − 0.167465 ΔCₜ(IGKC) + 0.683736 | | |
| Score15 = 0.531530 ΔCₜ(KIF20A) − 0.283226 | 0.7927883 | 0.83757907 |
| ΔCₜ(OSBPL1A) − 0.318978 ΔCₜ(CYP27A1) + | | |
| 1.933827 + 0.352991 ΔCₜ(AURKA) − 0.202966 | | |
| ΔCₜ(PTGER3) − 0.167651 ΔCₜ(IGKC) + 0.511166 | | |

1. A method for predicting the residual risk of recurrence after standard chemotherapy treatment, in particular a taxane-free chemotherapy treatment, and the benefit from inclusion of taxane in a chemotherapy regimen in a patient suffering from or at risk of developing recurrent neoplastic disease, in particular breast cancer, said method comprises the steps of:
(a) determining in a tumor sample from said patient the expression levels of the following 6 genes: UBE2C, KIF20A, PTGER3, OSBPL1A, CYP27A1, IGKC, and
(b) mathematically combining said expression level values for the genes of the said set which values were determined in the tumor sample to yield a prognostic combined score and
(c) comparing said prognostic combined score to one or more thresholds and classifying said patient in a good, intermediate or poor outcome group and
(d) determining in said tumor sample from said patient the expression levels of three genes: STC1, PCSK6, S100P, and
(e) mathematically combining said expression level values for STC1, PCSK6 and S100P to yield a predictive combined score, whereas a high predictive combined score generally indicates an increased likelihood of benefit from inclusion of taxane in a chemotherapy regimen in a patient classified to said poor and/or intermediate outcome group and a low combined score a decreased likelihood of benefit from inclusion of taxane in a chemotherapy regimen in a patient classified to said poor and/or intermediate outcome group.

2. The method of claim 1, wherein the expression levels of six genes: KIF20A, UBE2C, PTGER3, OSBPL1A, IGKC and CYP27A1 are used to calculate a predictive score, whereas a high combined score generally indicates an increased residual risk, of recurrence after standard chemotherapy treatment, and a low combined score a decreased residual risk of recurrence after standard chemotherapy treatment.

3. The method of claim 1, wherein the expression levels of three genes: S100P, PCSK6 and STC1 are used to calculate a predictive score, whereas a high combined score generally indicates an increased likelihood of benefit from inclusion of taxane in a chemotherapy regimen, and a low combined score a decreased likelihood of benefit from inclusion of taxane in a chemotherapy regimen.

4. The method of claim 1, wherein the markers are particularly suited for predicting residual risk of recurrence after standard chemotherapy treatment and the benefit from including a taxane to cytotoxic chemotherapy, preferably in estrogen receptor positive (ER+), Her2-negative (HER2−) tumors.

5. The method of claim 1, wherein said expression level is determined as an mRNA level.

6. The method of claim 1, wherein said expression level is determined by at least one of the following methods: a PCR based method, a microarray based method, or a hybridization based method, a sequencing and/or next generation sequencing approach.

7. The method of claim 1, wherein a preferred form is kinetic RT-PCP or quantitative reverse transcription polymerase chain reaction (qRT-PCR).

8. The method of claim 1, wherein said determination of expression levels is in a formalin-fixed paraffin-embedded tumor sample or in a fresh-frozen tumor sample.

9. The method of claim 1, wherein the expression level of said at least one marker gene is determined as a pattern of expression relative to at least one reference gene or to a computed average expression value.

10. The method of claim 1, wherein said step of mathematically combining the expression level values comprises a step of applying an algorithm to values representative of an expression level of a given gene.

11. The method of claim 1, wherein said algorithm is a linear combination of said values representative of an expression level of a given gene.

12. The method of claim 1, wherein a value for a representative of an expression level of a given gene is multiplied with a coefficient.

13. The method of claim 1, wherein one, two or more thresholds are determined for said combined scores and discriminated into response groups by applying the threshold on the combined score.

14. The method of claim 1, wherein one, two or more thresholds are determined for said gene expression level or combined scores and discriminated into (1) “predicted benefit” and “predicted non-benefit”, (2) “predicted benefit” and “predicted adverse effect”, (3) “predicted benefit”, “predicted indifferent effect” and “predicted adverse effect”, or more response groups with different probabilities of benefit by applying the threshold on the gene expression levels or the combined score.

15. The method of claim 1, wherein a high combined score is indicative of a benefit from taxane-based treatment.

16. The method of claim 1, wherein information regarding nodal status of the patient is processed in the step of mathematically combining expression level values for the genes to yield a combined score that predicts residual risk of recurrence after chemotherapy treatment.

17. A kit for performing the method of claim 1, said kit comprising a set of nine oligonucleotides of at least Seq ID Nos: 19, 20 or 21; Seq ID Nos: 16, 17, or 18; Seq ID Nos: 10, 11, or 12; Seq ID Nos: 13, 14, or 15; Seq ID Nos: 25, 26, or 27; Seq ID Nos: 22, 23, or 24; Seq ID Nos: 7, 8, or 9; Seq ID Nos: 4, 5, or 6; and Seq ID Nos: 1, 2, or 3; which oligonucleotides are capable of specifically binding sequences or to sequences of fragments of the genes in a combination of genes, wherein said combination comprises at least the 9 genes UBE2C, KIF20A, PTGER3, OSBPL1A, CYP27A1, IGKC, STC1, PCSK6 and S100P.

18. A method of using the kit of claim 17 comprising the steps of:
(a) determining in a tumor sample from said patient the expression levels of the following 6 genes: UBE2C, KIF20A, PTGER3, OSBPL1A, CYP27A1, IGKC, and
(b) mathematically combining said expression level values for the genes of the said set which values were determined in the tumor sample to yield a prognostic combined score and
(c) comparing said prognostic combined score to one or more thresholds and classifying said patient in a good, intermediate or poor outcome group and
(d) determining in said tumor sample from said patient the expression levels of three genes: STC1, PCSK6 and S100P, and
(e) mathematically combining said expression level values for STC1, PCSK6 and S100P to yield a predictive combined score, whereas a high predictive combined score generally indicates an increased likelihood of benefit from inclusion of taxane in a chemotherapy regimen in a patient classified to said poor and/or intermediate outcome group and a low combined score a decreased likelihood of benefit from inclusion of taxane in a chemotherapy regimen in a patient classified to said poor and/or intermediate outcome group.
